(19) United States
(12) Patent Application Publication
(10) Pub. No.: US 2014/0221782 A1
(43) Pub. Date: Aug. 7, 2014

(54) PREDICTION OF EXACERBATIONS FOR COPD PATIENTS

(71) Applicants: Ole Kristian Hejlesen, Hjorring (DK); Birthe Irene Dinesen, Aalborg (DK); Morten Hasselstrøm Jensen, Aalborg (DK); Simon Lebech Cichosz, Aarhus N (DK)

(72) Inventors: Ole Kristian Hejlesen, Hjorring (DK); Birthe Irene Dinesen, Aalborg (DK); Morten Hasselstrøm Jensen, Aalborg (DK); Simon Lebech Cichosz, Aarhus N (DK)

(73) Assignee: Aalborg Universitet, Aalborg Ø (DK)

(21) Appl. No.: 14/240,849

(22) PCT Filed: Aug. 24, 2012
(86) PCT No.: PCT/DK2012/050307

§ 371 Date: Feb. 25, 2014

(30) Foreign Application Priority Data
Aug. 26, 2011 (DK) PA 2011 00643
Oct. 11, 2011 (DK) PA 2011 00785

(51) Int. Cl. A61B 5/1455; A61B 5/08; A61B 5/0205

(52) U.S. Cl. CPC A61B 5/14552; A61B 5/0205; A61B 5/08
USPC 600/301; 600/323

(57) ABSTRACT

The invention provides a method for estimating if a patient suffering from Chronic Obstructive Pulmonary Disease (COPD) is approaching an exacerbation. Data with connected data indicative of levels of blood oxygen saturation obtained from the patient and their respective time of measurements are received, and a processor executes an algorithm involving: 1) calculating a statistical measure, e.g. a regression, of the level of blood oxygen saturation data taking into account available data within a time window with a limited length back in time, e.g. 30 days back in time, and 2) estimating if the patient is approaching an exacerbation by comparing a value obtained from said statistical measure to a reference value. Finally, an output indicative of a result of said estimation is generated. The method can ensure that COPD patients, e.g. in tele homecare, are properly treated before suffering from a severe exacerbation that could necessitate hospitalization. The COPD patient can easily measure blood oxygen saturation with an oxygen meter, and the algorithm can be executed on a mobile phone, a PC, on a server in contact with the patient via the interne, or on a dedicated oxygen meter device.

### FIELD OF THE INVENTION

The present invention relates to field of medical devices, more specifically the invention provides an algorithm for prediction or forecasting exacerbations for COPD patients, and a system for performing such prediction or forecasting.

### BACKGROUND OF THE INVENTION

Today, many patients suffering from Chronic Obstructive Pulmonary Disease (COPD) live in their homes, and they are they only contact a medical staff when they have an exacerbation which requires medicine or other kind of treatment therapy. An exacerbation is an acute event with a significant worsening of lung function and symptoms. Patients who receive proper therapy rapidly after the onset of symptoms have much better outcomes than those who wait several days.

A median frequency of exacerbations in COPD patients is 2-3 per year, and today a typical COPD patient visits a medical doctor once a year, where a prediction of exacerbation risk for the patient is evaluated using e.g. the so-called BODE index including the patient's body mass index to predict the annual number of exacerbations and their severity.

However, an annual evaluation of each patient is not sufficient to avoid severe exacerbations in practice. It turns out that many patients tend to seek assistance too late after having suffered several days from symptoms of an exacerbation, and thus they end in a medical state requiring hospitalization.

In Denmark, COPD exacerbations cause about 23,000 hospitalizations each year. This is burden for the health care system, since hospitalization of a patient is expensive compared to alternative treatments, such as the patient being in telephone contact with a doctor ordering medicine etc. Furthermore, having a severe exacerbation is unpleasant for the patient, and weaker patient may even die as a cause of the exacerbation.

### SUMMARY OF THE INVENTION

Following the above description there is a need for finding a way to help COPD patients to avoid exacerbations, and thus avoid hospitalizations. Thus, it may be seen as an object of the present invention to provide a method and a system for helping COPD patients to avoid or at least reduce the number of exacerbations.

In a first aspect, the invention provides a method for estimating if a patient suffering from Chronic Obstructive Pulmonary Disease is approaching an exacerbation, the method comprising
receiving a set of data with connected data indicative of levels of blood oxygen saturation obtained from the patient and their respective time of measurements,
executing an algorithm on a processor, the algorithm involving
calculating a statistical measure, such as a regression, of the level of blood oxygen saturation data taking into account available data within a time window with a limited length back in time, such as using a time window with a length of 30 days, and
estimating if the patient is approaching an exacerbation by comparing a value obtained from said statistical measure to a reference value, and
generating an output indicative of a result of said estimation.

The invention is based on the insight, that it is possible to reliably predict an exacerbation of in a COPD patient solely by having a series of measurements of blood oxygen saturation, and their time of measurements. Preferably, measurement representing a period of one or more days back in time, e.g. 20-40 days back in time, has proven on a population of COPD patients to provide a reliable prediction of exacerbations. Thus, by means of simple measurements, e.g. performed by the patient once a day, or only once a week, provides useful data to allow a moving prediction type of automatic prediction if the patient, at a given time, is approaching an exacerbation or not. Hereby, a proper therapy can be initialized before the symptoms reach a level where the patient needs to be hospitalized. Either only the patient is warned about an approaching exacerbation, and/or a medical doctor and/or relatives to the patient can be warned as well, if the outcome of the algorithm has determined that the patient approaches an exacerbation.

The method is advantageous since it can be utilized with low cost equipment, since only one medical instrument, an oxygen meter, is necessary. Such device can easily be operated by the patient in the patient's home, and either blood oxygen data can be automatically or manually transferred to another device with a processor executing the prediction algorithm. Such other device with a processor executing the algorithm mobile may be a mobile phone (smart phone), a laptop PC, a tablet or the like, or it may be a server located at a hospital or at another location. Thus, the method is highly suited for being utilized as a tele homecare solution. However, it is to be understood that the method could also be used for patients being hospitalized.

It has been proven that it is possible to reliably prediction a risk for an approaching exacerbation solely based on measured levels of blood oxygen saturation, and thus in some embodiments, only this parameter is taken into account. However, it is to be understood that it is possible to provide a combined prediction based on blood oxygen level measurements together with one or more measured medical conditions of the patient and/or further information about the patient. Hereby the exacerbation prediction can be made even better.

The algorithm may be seen as a ‘moving prediction’ of exacerbations, where a prediction is performed e.g. on a day-to-day basis. This is new and advantageous compared to the known BODE method which is not useful as a tool for avoiding exacerbations. A window of for example 30 days is moved every day and the data in the past 30 days are used to make the prediction. The reason why this “prediction resolution” can be increased is due to the data used. Former prediction attempts concerned less dynamic data like body master index, forced expiratory volume in 1 second (FEV1) and dyspnea scale, while the attempt according to the invention concerns the dynamic data oxygen saturation, e.g. combined with one or both of: pulse and blood pressure, which show day-to-day variations.

Based on the output from the method—to the patient and/or relatives to the patient and/or medical personnel, it is possible to initiate proper therapy, e.g. antibiotic medicine, if the method reveals that an exacerbation is approaching. Hereby, the patient can feel safe, and even if it is predicted that an exacerbation is approaching, a therapy can be initiated before the patient has a severe discomfort of an exacerbation, and hospitalization can thus often be avoided.

The method according to the invention has been validated based on the so-called TELEKAT project with 111 COPD patients of who 57 were equipped with measurement equipment. The TELEKAT project was funded by the Program for User-driven Innovation, the Danish Enterprise and Construction Authority, the Center for Healthcare Technology at Aalborg University, and by various clinical and industrial partners in Denmark. See e.g. the paper “Moving prediction of exacerbation in chronic obstructive pulmonary disease for patients in telecare” by the inventors, Journal of Telemedicine and Healthcare Vol. 18, No. 2, 2012.

In the following different embodiments will be mentioned.

The time window may be a time window which weights all blood oxygen saturation measurements within the window length equally, i.e. a square time window. However, the time window may alternatively weight older measurements less in relation to newer measurements, such as the time window applying an exponentially decaying weighting of level of blood oxygen saturation data back in time. Hereby, it is possible to put more weight on the latest developments in blood oxygen levels, which may improve the performance of the prediction.

The time window may have a length of 1-90 days, 2-60 days, such as 10-50 days, such as 15-40 days. More specifically, the time window may have a length of 25-35 days, such as a length of 30 days. The selected length of time window may be made independent of the individual patient, e.g. dependent on the expected interval between blood oxygen saturation measurements. A long time window can be used for rather healthy patients who only measure blood oxygen saturation once or twice a week, while a shorter time window, down to a couple of days may be used for patients performing one or more measurements per day.

In a preferred, simple embodiment, calculating the statistical measure comprises calculating a regression of the level of oxygen saturation data within the time window. Especially, such regression calculation may be a linear regression calculation, which is very simple to calculate. However, the regression may also be a non-linear regression, e.g. a polynomial regression. In all cases, a regression of the available blood oxygen saturation measurements within the time window versus their measurement times are used for calculating a regression as a statistical measure. A linear regression has proven to provide a reliable prediction result, but other types of regression may be used as well.

In case of a linear regression, the output may be generated based on a comparison of a slope of the calculated linear regression with a reference value, such as a reference value determined for the individual patient.

The method may comprise calculating a statistical measure comprising at least one of: variance, mean, skewness, and kurtosis. E.g. such measure(s) may be used to supplement a regression measure, so as to improve reliability of the prediction method even further.

The output may be generated taking into account further data indicative of information related to the patient, such as age, sex or one or more medical conditions. E.g. such information may be taken into account in selection of the reference value, e.g. a reference value used for comparison with a calculated regression slope value. Further, e.g. age may be taken into account in selecting how to inform the patient of the prediction result. E.g. for very old patients, a green or red lamp can be used to indicate “No exacerbation risk” and “Exacerbation approaching”, whereas younger patient may be informed by an SMS on their mobile phone, via an email or the like. Especially, the information related to the patient comprises data indicative of a medical condition of the patient. More specifically, the data may comprise data representing a measured value indicative of: blood pressure, heart rate, and lung function such as FEV1. Especially, heart rate can easily be determined, since many optical oxygen meters which can provide blood oxygen level data at the same time also measure the patient's heart rate. Thus, in some embodiments, heart rate can be used to supplement the prediction based on blood oxygen level measurements and provide a prediction which is even more reliable.

The method may comprise performing a binary decision if the patient is approaching an exacerbation or not, based on said comparison with a reference level, and wherein the output is indicative of a binary decision, e.g. “An exacerbation is approaching” or “No exacerbation approaching”. Alternatively, the method may comprise calculating a risk or a certainty value indicative of a graduated risk or certainty of the patient approaching an exacerbation, and wherein the output is indicative of said risk or certainty value.

In some embodiments, the algorithm is executed, preferably automatically, and the output is generated, when a new level of blood oxygen saturation data is received, such as when a new level of oxygen saturation data is entered by the patient. In a variant, an alarm device, e.g. a mobile phone, is used to inform the patient that it is time to do a measurement.

The output, and thus the result of the prediction, may is preferably made available to the patient and/or to medical personnel and/or relatives to the patient. Younger, rather healthy patients, can be informed themselves, while relatives and/or medical personnel can be informed in case of older, rather weak, patients.

It may be preferred to adjust at least the calculating step or the estimating step of the algorithm in response to one or more parameters related to the individual patient. Hereby, it may be possible to improve reliability of the method even further. Especially, said one or more parameters related to the individual patient comprises at least one of: a length of the time window, said reference value, and selection of possible further data to be included in the algorithm, such as data indicative of heart rate and/or blood pressure of the patient.

The method may in practice be implemented by means of a computer executable program code stored on a storage medium, wherein the code is arranged to perform the method according to the first aspect, when being executed on a device comprising a processor.

In a second aspect, the invention provides an apparatus comprising a processor arranged to perform the method according to the first aspect. Especially, it is preferred that the apparatus comprises an oxygen meter arranged to measure a level of blood oxygen saturation of a patient and to provide data according to a measured level of blood oxygen saturation. In principle any type of blood oxygen meters can be used, however optical types of oxygen meters using a clip to mount on a finger can easily be operated by the patient, and thus such meters are preferred for tele homecare systems. More specifically, the oxygen meter may further be arranged to measure a heart rate of the patient. This is often automatically done by optical type of oxygen meters, and thus such meters allow to take into account both oxygen saturation data as well as heart rate in performing a exacerbation risk prediction based on statistical data.

The apparatus may comprise a meter device arranged to measure data indicative of at least one further medical condition of the patient apart from blood oxygen saturation. Such additional medical condition may be heart rate, as just mentioned.

The apparatus may be implemented in various ways with known components, as will be illustrated by example embodiments in the following.

The apparatus may comprise an output indicator, such as a visual output indicator comprising a display and/or a colored light, so as to generate an output indicative of the result of said estimation. A display may indicate the prediction result in a color, in text, in a number between 0 and 1, or a % value between 0 and 100, or any combination of these. A colored light, e.g. red and green may be used to indicate a “exacerbation approaching”, “no exacerbation approaching”, respectively. In one embodiment, the oxygen meter, and the output indicator are housed so as to form one single unit, such as housed within one single casing. This can be implementation of the prediction algorithm in software in an existing oxygen meter which often has a display which can then be used to output the prediction result. In such case, a processor within the oxygen meter is used to execute the prediction algorithm.

In another embodiment, the oxygen meter and the processor are part of separate units, such as being housed within respective separate casings, wherein the separate units are functionally connected by a wireless or a wired connection. E.g. the oxygen meter can wirelessly transmit measured blood oxygen saturation data to a mobile phone or a computer which then executes the prediction algorithm.

Thus, in some embodiments, the unit comprising the processor is one of: a mobile phone, a personal computer, and a dedicated device such as an oxygen meter.

In some embodiments, the unit comprising the processor also comprises an output indicator arranged to generate an output indicative of the result of said estimation. This could also be obtained with a mobile phone, a personal computer, or a dedicated device such as an oxygen meter.

In some embodiments, the unit comprising the oxygen meter is functionally connected to the unit comprising the processor via the internet, such as via a personal computer or a mobile phone.

The apparatus may comprise a user interface so as to allow manually entering of measured levels of blood oxygen saturation. Such embodiment can use an existing oxygen meter in combination with a computer or a mobile phone on which a measured value is entered, and the processor in the computer or mobile phone then executes a program code implementing the predicting algorithm based on the latest entered value together with previously entered and stored values.

The apparatus may comprise an output indicator arranged to provide an output indicative of the result of said estimation to the patient and an output indicator arranged to provide an output indicative of the result of said estimation to medical personnel, preferably the apparatus comprises an output indicator to provide an output indicative of the result of said estimation to one or more relative to the patient.

The apparatus may comprise an alarm functionality serving to automatically alarm medical personnel, in case the output indicative of the result of said estimation indicates an exacerbation.

It may be preferred that the oxygen meter is arranged for operation by the patient in his/her home, and wherein the system comprises an output indicator arranged to provide the output indicative of the result of said estimation to one or more of: medical personnel at a hospital, a clinical centre, a general practitioner, and a relative to the patient.

Especially, the apparatus may be in the form of a tele homecare system, wherein the oxygen meter is arranged for operation by the patient in his/her home, and wherein the system comprises an output indicator arranged to provide the output indicative of the result of said estimation to one or more of: medical personnel at a hospital, a clinical centre, a general practitioner, and a relative to the patient. The algorithm may be executed at a processor either in the patient's home or at a server at a remote destination, such as a hospital server or the like.

The first and second aspects may be combined and their respective embodiments intermixed with each other. Further, the mentioned advantages for the first aspect apply as well for the second aspect. These and other aspects of the invention will be apparent from and elucidated with reference to the embodiments described hereinafter.

### BRIEF DESCRIPTION OF THE FIGURES

Embodiments of the invention will be described in more detail in the following with regard to the accompanying figures. The figures show one way of implementing the present invention and is not to be construed as being limiting to other possible embodiments falling within the scope of the attached claim set.

FIG. 1 shows a block diagram of one embodiment,

FIG. 2 shows a block diagram of a possible implementation with two separate devices,

FIG. 3 shows a block diagram of a possible implementation with one single dedicated device, and

FIG. 4 shows a block diagram of a possible implementation involving three separate devices.

### DETAILED DESCRIPTION OF AN EMBODIMENT

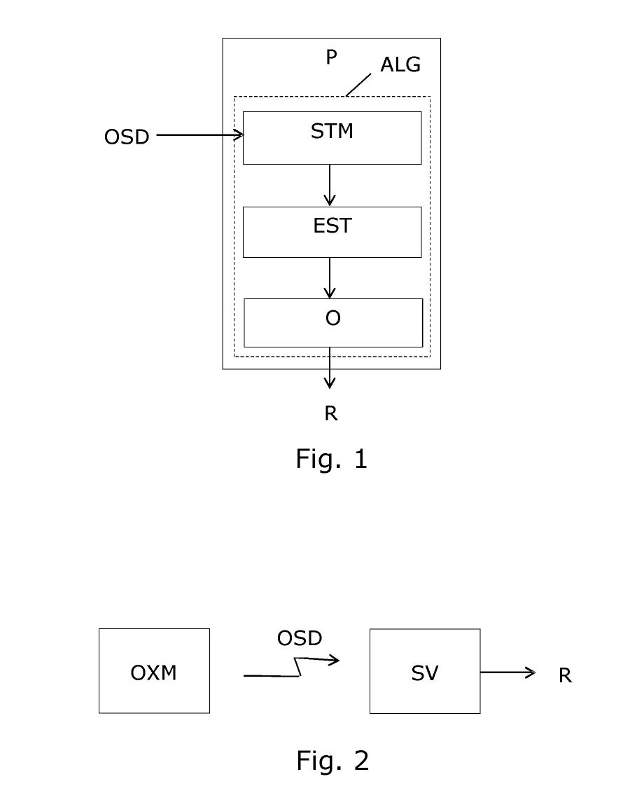

FIG. 1 shows a block diagram of an embodiment with basic parts of an implementation of the method according to the invention. A processor P, e.g. in a mobile phone or a computer, has a software implementing an exacerbation prediction algorithm ALG. Blood oxygen saturation data OSD including measured blood oxygen saturation level values and their time of measurement are received and entered into the algorithm ALG, e.g. manually entered or automatically transmitted by wire or wirelessly to the device including the processor P. The blood oxygen measurements can be performed by the COPD patient in his/her home, but it may also be performed e.g. by medical personnel at a hospital.

The algorithm ALG comprises the step of calculating a statistical measure STM of the blood oxygen saturation data OSD taking into account available data a limited length back in time. A preferred statistical measure is a measure related to a regression, e.g. a linear regression. A time window with a length of 1-90 days, e.g. 30 days back in time can preferably be used. A preferred statistical measure is a slope determined by calculating a linear regression of the available data within the time window. The calculated statistical measure is then provided to an estimation EST involving estimating if the patient is approaching an exacerbation by comparing a value obtained from said statistical measure to a reference value, e.g. the slope as mentioned, and finally an output indicative of the result of the estimation EST is generated as an output O, e.g. as text on a display, a visual or audible alarm etc.

As an example, the statistical measure F_{sat,I.reg,[30;0]} is a linear regression of oxygen saturation data 30 days before the event. More specifically, a linear regression is calculated on oxygen saturation data and a linear expression is produced: y=ax+b. The coefficient a, which is the slope of the line, is used as the predictor. When an exacerbation is impending a tends to decrease, i.e. oxygen saturation is decreasing. Since the best classifier only counts this feature, a simple mathematical expression for the classifier can be expressed:

$$f ( a ) = \{ \begin{matrix}1 , & a < {a}_{0} \\ 0 , & a \geq {a}_{0} ,\end{matrix} \\ {a}_{0} = - 0.0737$$

This expression outputs an indication of exacerbation (f=1) or not (f=0) based on the inputted coefficient a.

As a specific example to illustrate the principle: a man has measured his oxygen saturation every week during the last month (four weeks): [97% 98% 96% 95%]. The slope a of the linear regression is calculated to −0.8:

$$f ( - 0.8 ) = \{ \begin{matrix}1 , & - 0.8 < - 0.0737 \\ 0 , & - 0.8 \geq - 0.0737\end{matrix} ⇔ f ( - 0.8 ) = 1$$

The classifier indicates that the man gets an exacerbation, and an output can be generated accordingly.

It is to be understood that the found statistical measure or feature is not unambiguous. For example polynomial regression may be an alternative, an may produce even more reliable prediction results. Other statistical measures for example skewness and kurtosis might also produce useful results in the same interval. Finally, other physiological data like blood pressure and pulse (heart rate) are candidates for supplementing the prediction.

The chosen window length of 30 days has been shown to produce reliable results on the available test data and is thus based on data resolution in the test data with only one measurement pr. week and the minimum number of data points for the calculation of mean, std, linear regression, skewness and kurtosis was defined as 3. A shorter time window length, 2-10 days, or the like, would give more specific predictions. Furthermore, the form of the window could be changed. The window may be a square window, meaning that each measurements is weighed equally, but a window where older measurements are weighed less in relation to newer measurements would be a possible alternative which may produce even better results.

The model/classifier can be optimized to all the patients in a study population. However, inter-patient variability is often observed and a classifier fitted to the individual patient would in that case yield better results. This demands more patients and more data pr. patient. In modelling this issue is referred to as estimation of global and patient specific parameters.

FIG. 2 shows a possible implementation with at least two separate devices. An oxygen meter OXM transmits measured oxygen saturation data OSD e.g. via the internet, to a server SV with has a processor and software executing the algorithm according to the invention. The server the outputs the prediction result R.

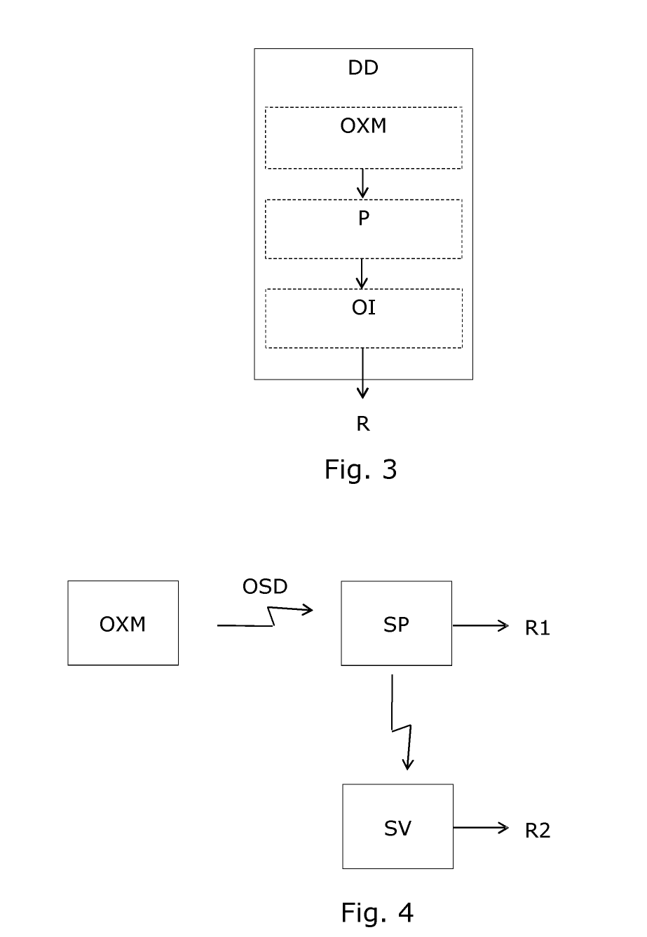

FIG. 3 shows an alternative implementation with a dedicated device DD which, within one casing, includes an oxygen meter OXM, a processor P with program code adapted to perform the algorithm according to the invention, and an output indicator OI, e.g. a display for showing the prediction result. Such dedicated device DD may be in the form of an oxygen meter which has been modified to execute software to implement the method of the invention and to display the prediction result on a display or by means of light emitting diodes etc.

FIG. 4 shows yet another implementation involving three devices. An oxygen meter OXM transmits measured oxygen saturation data OSD to a smart phone which performs the prediction and outputs a result to the patient R1. The smart phone transmits the result, or the OSD data to a server SV which outputs a result R2, e.g. to a medical doctor at the hospital.

It is to be understood that there are several other variants possible for implementation of the invention using combinations of know devices. Thus, the invention is easy to implement in practice, e.g. in a tele homecare solution using known low cost components.

To sum up, the invention provides a method for estimating if a patient suffering from Chronic Obstructive Pulmonary Disease (COPD) is approaching an exacerbation. Data with connected data indicative of levels of blood oxygen saturation obtained from the patient and their respective time of measurements are received, and a processor executes an algorithm involving: 1) calculating a statistical measure, e.g. a regression, of the level of blood oxygen saturation data taking into account available data within a time window with a limited length back in time, e.g. 30 days back in time, and 2) estimating if the patient is approaching an exacerbation by comparing a value obtained from said statistical measure to a reference value. Finally, an output indicative of a result of said estimation is generated. The method can ensure that COPD patients, e.g. in tele homecare, are properly treated before suffering from a severe exacerbation that could necessitate hospitalization. The COPD patient can easily measure blood oxygen saturation with an oxygen meter, and the algorithm can be executed on a mobile phone, a PC, on a server in contact with the patient via the internet, or on a dedicated oxygen meter device.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A method for estimating if a patient suffering from Chronic Obstructive Pulmonary Disease is approaching an exacerbation, the method comprising:
receiving a set of data with connected data indicative of levels of blood oxygen saturation obtained from the patient and their respective time of measurements,
executing an algorithm on a processor, the algorithm involving
calculating a statistical measure of the level of blood oxygen saturation data taking into account available data within a time window with a limited length back in time, and
estimating if the patient is approaching an exacerbation by comparing a value obtained from said statistical measure to a reference value, and
generating an output indicative of a result of said estimation.

2-33. (canceled)

34. The method according to claim 1, comprising calculating a regression of the level of oxygen saturation data within the time window.

35. The method according to claim 34, wherein the regression calculation is a linear regression calculation, and
wherein the output is generated based on a comparison of a slope of the calculated linear regression with a reference value.

36. The method according to claim 1, wherein the time window has a length of 1-90 days.

37. The method according to claim 1, wherein the output is generated taking into account further data indicative of information related to the patient, wherein said information related to the patient comprises data indicative of a medical condition of the patient comprising data representing a measured value indicative of: blood pressure, heart rate, and lung function.

38. The method according to claim 1, comprising performing a binary decision if the patient is approaching an exacerbation or not, based on said comparison with a reference level, and wherein the output is indicative of a binary decision.

39. The method according to claim 1, comprising calculating a risk or a certainty value indicative of a graduated risk or certainty of the patient approaching an exacerbation, and wherein the output is indicative of said risk or certainty value.

40. The method according to claim 1, wherein the algorithm is executed and the output is generated, when a new level of blood oxygen saturation data is received.

41. The method according to claim 1, comprising adjusting at least the calculating step or the estimating step of the algorithm in response to one or more parameters related to the individual patient comprising at least one of: a length of the time window, said reference value, or selection of possible further data to be included in the algorithm.

42. An apparatus comprising a processor arranged to perform the method according to claim 1.

43. The apparatus according to claim 42, comprising an oxygen meter arranged to measure a level of blood oxygen saturation of a patient and to provide data according to a measured level of blood oxygen saturation.

44. The apparatus according to claim 42, comprising a meter device arranged to measure data indicative of at least one further medical condition of the patient apart from blood oxygen saturation.

45. The apparatus according to claim 42, comprising an output indicator, so as to generate an output indicative of the result of said estimation.

46. The apparatus according to claim 45, wherein the processor, an oxygen meter, and the output indicator are housed so as to form one single unit.

47. The apparatus according to claim 42, wherein an oxygen meter and the processor are part of separate units, wherein the separate units are functionally connected by a wireless or a wired connection.

48. The apparatus according to claim 47, wherein the unit comprising the processor is a mobile phone, a personal computer, a dedicated device or an oxygen meter.

49. The apparatus according to claim 42, comprising a user interface so as to allow manually entering of measured levels of blood oxygen saturation.

50. The apparatus according to claim 42, comprising an alarm functionality serving to automatically alarm medical personnel, in case the output indicative of the result of said estimation indicates an exacerbation.

51. The apparatus according to claim 42, comprising an oxygen meter, which is arranged for operation by the patient in his/her home, and wherein the system comprises an output indicator arranged to provide the output indicative of the result of said estimation to one or more of: medical personnel at a hospital, a clinical centre, a general practitioner, or a relative of the patient.

52. A computer executable program code stored on a storage medium, wherein the code is arranged to perform the method according to claim 1, when being executed on a device comprising a processor.
